(19) United States
(12) Patent Application Publication
(10) Pub. No.: US 2026/0183188 A1
(43) Pub. Date: Jul. 2, 2026

(54) ENHANCED SAUNA DEVICE

(71) Applicant: Sauna Works Inc. (aka Far Infrared Sauna Technology Co.), Berkeley, CA (US)

(72) Inventors: Raleigh C. Duncan, Berkeley, CA (US); Andrew Kaps, San Francisco, CA (US)

(73) Assignee: Sauna Works Inc. (aka Far Infrared Sauna Technology Co.), Berkeley, CA (US)

(21) Appl. No.: 19/008,529

(22) Filed: Jan. 2, 2025

(51) Int. Cl. A61H 33/06; A61F 7/00; A61H 33/00

(52) U.S. Cl. CPC A61H 33/066; A61F 7/0097; A61H 33/005; A61H 33/063; A61F 2007/0093; A61H 2033/061

(57) ABSTRACT

A convertible heat therapy system is disclosed, which may be utilized in two distinct configurations to enhance user experience. According to various embodiments, the system can be deployed as a sauna blanket, providing focused heat application, or alternatively, it may be transformed into a sauna dome, enveloping the user in a warm enclosure. The device may include, in certain embodiments, features such as sensors and controllers and intelligently selected materials to enhance comfort and heat delivery, thereby potentially offering improved therapeutic benefits and versatility in personal heat therapy applications.

### TECHNICAL FIELD

The present disclosure relates generally to sauna devices, and more specifically to enhanced multipurpose sauna devices.

### DESCRIPTION OF RELATED ART

Infrared sauna blankets have emerged as a popular trend in personal wellness and heat therapy. These portable, wrap-around designs aim to provide users with the benefits of infrared radiation, including deep tissue heating, detoxification through sweating, relaxation, and pain relief. However, despite their convenience and initial appeal, conventional infrared sauna blankets suffer from significant inadequacies that compromise their effectiveness and user experience.

When a sauna blanket is used as a cover on top of the body, a substantial portion of the emitted infrared radiation is absorbed by the blanket's own materials or reflected back onto the device, rather than penetrating deep into the body tissues. This inefficiency diminishes the intended therapeutic benefits. Furthermore, to prevent burns and ensure safety, users are often advised to wear light, breathable clothing while using infrared sauna blankets. Paradoxically, this protective measure also acts as an insulating barrier, further reducing the amount of infrared radiation that can effectively reach and warm the body.

Conventional sauna blankets typically rely on a flat, two-dimensional design that cannot conform to the complex contours of the human body. This leads to uneven heat distribution, with some areas receiving excessive heat while others remain under-heated. The wrapped configuration can feel claustrophobic or restrictive for some users, limiting mobility and overall comfort during the therapy session. This discomfort may shorten the effective treatment time, undermining the potential benefits. Consequently, it is desirable to provide enhanced sauna devices that overcome at least some of the drawbacks of conventional mechanisms.

### BRIEF DESCRIPTION OF THE DRAWINGS

The disclosure may best be understood by reference to the following description taken in conjunction with the accompanying drawings, which illustrate particular embodiments.

FIG. 1 illustrates an example of an exterior side of a combination sauna dome sauna blanket.

FIG. 2 illustrates an example of an interior side of a combination sauna dome sauna blanket.

FIG. 3 illustrates an example of the combination device in a sauna dome mode.

FIGS. 4a-4b illustrates an example of the different layers of the combination sauna dome sauna blanket in sauna blanket mode and in sauna dome mode.

FIG. 5 illustrates a process flow of a technique for controlling the combination device.

FIG. 6 illustrates one example of a controller, configured in accordance with various embodiments.

### DESCRIPTION OF EXAMPLE EMBODIMENTS

Reference will now be made in detail to some specific examples including the best modes contemplated by the inventors. Examples of these specific embodiments are illustrated in the accompanying drawings. While various embodiments are disclosed herein, it will be understood that they are not intended to be limiting. On the contrary, they are intended to cover alternatives, modifications, and equivalents as may be included within the spirit and scope of the disclosure as defined by the appended claims.

For example, the techniques will be described in the context of sauna blankets, and heating elements associated with such sauna blankets. However, it should be noted that the techniques apply to a wide variety of different environments and devices. In the following description, numerous specific details are set forth in order to provide a thorough understanding of the present invention. Particular example embodiments may be implemented without some or all of these specific details. In other instances, well known process operations have not been described in detail in order not to unnecessarily obscure the present disclosure.

Various techniques and mechanisms of the present disclosure will sometimes be described in singular form for clarity. However, it should be noted that some embodiments include multiple iterations of a technique or multiple instantiations of a mechanism unless noted otherwise. For example, a system uses a processor in a variety of contexts. However, it will be appreciated that a system can use multiple processors while remaining within the scope of the present disclosure unless otherwise noted. Furthermore, the techniques and mechanisms of the present disclosure will sometimes describe a connection between two entities. It should be noted that a connection between two entities does not necessarily mean a direct, unimpeded connection, as a variety of other entities may reside between the two entities. For example, a processor may be connected to memory, but it will be appreciated that a variety of components may reside between the processor and memory. Consequently, a connection does not necessarily mean a direct, unimpeded connection unless otherwise noted.

### Overview

A convertible heat therapy system is disclosed, which may be utilized in two distinct configurations to enhance user experience. According to various embodiments, the system can be deployed as a sauna blanket, providing focused heat application, or alternatively, it may be transformed into a sauna dome, enveloping the user in a warm enclosure. The device may include, in certain embodiments, features such as sensors and controllers and intelligently selected materials to enhance comfort and heat delivery, thereby potentially offering improved therapeutic benefits and versatility in personal heat therapy applications.

### EXAMPLE EMBODIMENTS

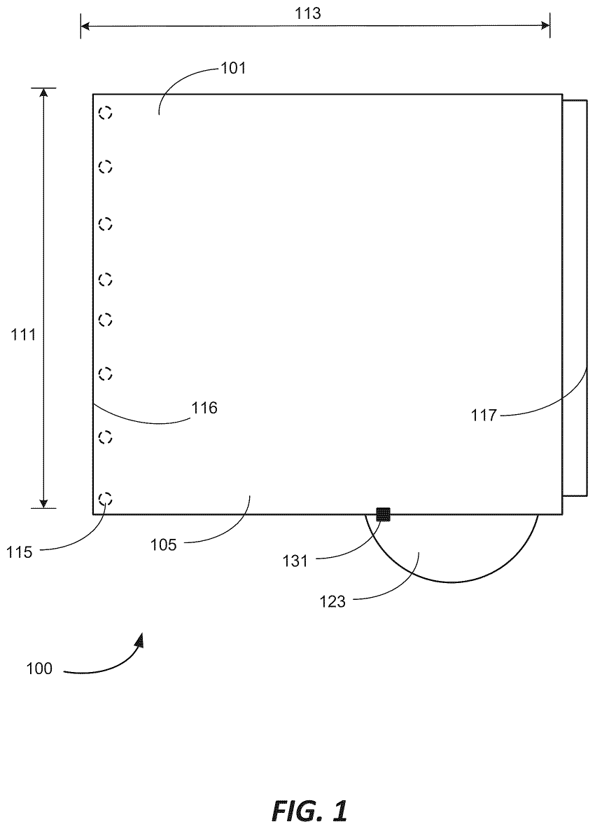

FIG. 1 illustrates an example of an exterior side 101 of a combination sauna dome sauna blanket 103, also referred to herein as a combination device or sauna dome sauna blanket. According to various embodiments, the combination sauna dome sauna blanket 103 is a flexible, thermally insulating structure or cover that may resemble a large heavy blanket that provides users with a convenient heat therapy system. In particular implementations, the device facilitates easy transformation between at least two distinct modes: a sauna blanket for intimate, contact warmth, and a sauna dome for an immersive, enclosed heat environment without significant contact with the user.

In a particular example, the exterior side 101 of the combination device when in sauna dome mode features a durable, water-resistant polyurethane (PU) leather material 105, allowing ease of cleaning and maintenance. According to various embodiments, the dimensions of the device are selected to accommodate users of varying heights, with a sauna dome sauna blanket length 111 of approximately 50-80 inches and a blanket width 113 of 65-100 inches. In sauna dome mode, the height of the dome or radius of the substantially semicylindrical compartment can be approximately 25-35 inches. In another embodiment, the height of the dome is approximately 10-25 inches. The length of the sauna dome may be anywhere from 50-80 inches. The width of the sauna may be approximately 20-30 inches. The sizing can also be customized for particular users. The sauna dome need not be strictly semicylindrical, but can be substantially semicylindrical allowing for a rounded or elliptical curvature on the upper portion of the sauna dome to accommodate a human body. The sauna dome upper portion can also be hexagonal as an alternative to semicylindrical in shape.

To facilitate easy transformation between modes, magnets may be discreetly integrated along the edge 115 of the device. In particular implementations, these magnets securely connect to the opposite edge 117 of the device when in sauna dome mode, ensuring a snug and stable enclosure. According to various embodiments, a head end cover 123 at the head end of the device may help prevent heat from escaping during sauna dome operation.

According to various embodiments, alternative materials and dimensions can be employed to cater to diverse user preferences and needs. For instance, eco-friendly options like recycled polyester or plant-based synthetic leather might replace traditional PU leather in certain implementations. In other cases, the device's dimensions can be adjusted to accommodate taller or shorter individuals, or even designed for multiple individuals or shared use.

The combination sauna dome sauna blanket 101 offers numerous benefits, including enhanced versatility, convenience, and cost-effectiveness. According to various embodiments, this single device provides two distinct heat therapy experiences.

According to various embodiments, the sauna dome sauna 101 includes a power socket such as a 6 pin socket 131 designed to establish a secure connection with a power source. According to various embodiments, this socket is carefully configured to ensure the safe and efficient delivery of power to the device's heating elements and controller.

In particular implementations, the 6-pin socket 131 includes a specific pin layout, with each pin serving a distinct purpose. The first pin, VCC, delivers the primary power supply, typically +12V DC, while the second pin, GND, provides a secure grounding connection. The remaining pins facilitate various functions: CTRL for receiving control signals, SENS for accommodating sensor inputs, CLK for transmitting clock signals, and RSV, reserved for future expansions or proprietary uses.

Overcurrent protection (OCP), short-circuit protection (SCP), and ground fault circuit interrupter (GFCI) protection may be integrated into the socket or power supply unit (PSU) to prevent electrical shock, damage, or other hazards.

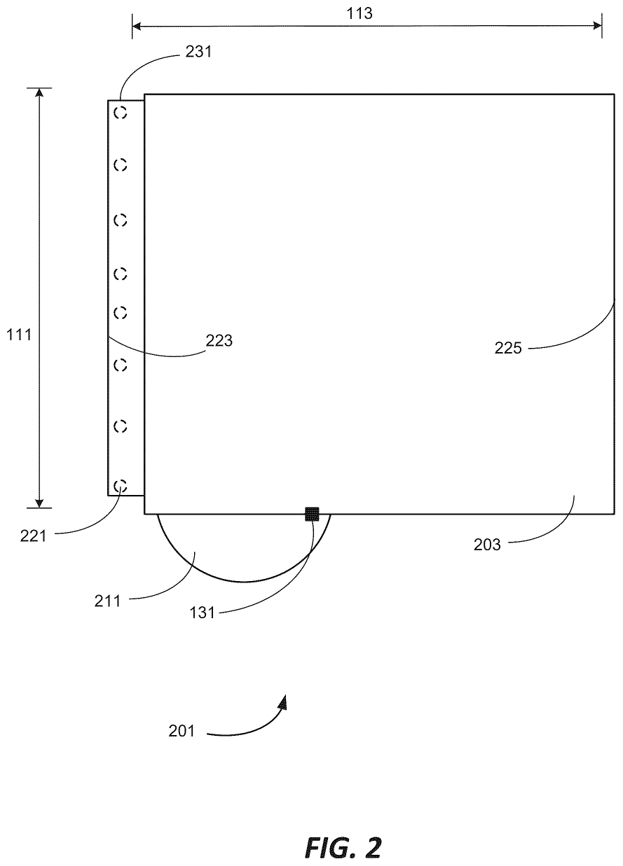

FIG. 2 illustrates one example of an interior side of the sauna dome sauna blanket. In accordance with various embodiments, the interior side 201 of the combination sauna dome sauna blanket 203. In particular embodiments, the interior side is constructed of a PU (Polyurethane) leather material, offering a unique blend of benefits for this specific application including softness, comfort, easy cleaning, simple maintenance, prevention of fungal growth, moisture resistance, abrasion resistance, and thermal insulation.

In particular embodiments, one or more end covers 211 or flaps are situated at that head and or foot ends of the device. The one or more end covers 211 may operate to maintain thermal integrity and prevent heat escape when the device is operated in sauna dome mode. This end cover 211 can be attached to the sauna dome as separate pieces or as a flap using a variety of different mechanisms.

According to various embodiments, multiple magnets 221, e.g. 8-10, are position at the edge 223 of the interior side of the device and are designed to interact with correspond metal attachment elements situated on the opposite edge 225 of the device when in sauna blanket mode such that when folded into sauna dome mode, the magnetic coupling system allows effortless and secure attachment of the folded blanket section to the upper cover of the sauna dome, thereby providing a snug and thermally efficient enclosure.

In particular embodiments, the portion 231 of the sauna blanket that is folded into the upper cover of the sauna dome measures approximately 40-50 or 43 inches in width, providing an ample and comfortable interior space for users.

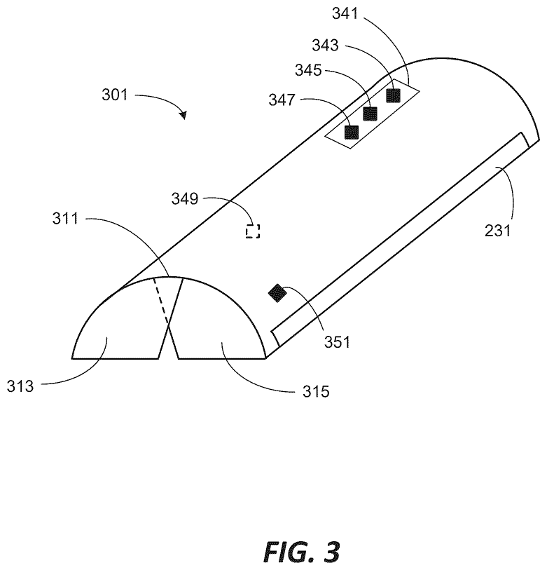

FIG. 3 illustrates one example of a combination device in a sauna dome mode, showcasing the transformative design of the device. In particular embodiments, the sauna dome 301 is depicted with a head end cover 311, featuring flaps 313 and 315 that may be constructed from PU leather, offering enhanced durability and ease of maintenance. Each flap, 313 and 315, incorporates one or more metal sheets 317 and 319, respectively, which can facilitate secure placement and efficient heat retention.

In particular implementations, the foot end cover can be attached, made of flaps, or may be a separate module piece than is attached via zipper or Velcro or other mechanisms.

According to various embodiments, the combination device in sauna dome mode exhibits specific dimensions, with a height of approximately 12.6 inches and a width of approximately 34 inches. These calibrated dimensions can accommodate users of varying heights, ensuring a comfortable and immersive heat therapy experience.

In various embodiments, the end covers and flaps may be designed with different materials, such as eco-friendly alternatives to PU leather, or innovative textiles offering enhanced thermal insulation. Additionally, the dimensions of the sauna dome mode can be adjusted to cater to diverse user preferences, including taller or shorter individuals, or even designed for multiple users or shared use.

In particular embodiments, the flaps 313 and 315 may feature adjustable mechanisms, allowing users to customize the level of heat retention and airflow within the sauna dome. Furthermore, integrated sensors 341 and controller 351 may be employed to monitor and adjust temperature, humidity, and other environmental factors, providing a personalized and optimal heat therapy experience.

According to Various Embodiments, the Combination Device in Sauna Dome Mode May incorporate integrated sensors 341, strategically positioned to monitor a range of environmental factors crucial for an optimal heat therapy experience. In particular implementations, these sensors 341 can include, but are not limited to, temperature sensors 343 (e.g., thermistors or thermocouples) to track the internal temperature of the sauna dome, humidity sensors 345 (e.g., hygrometers) to measure the ambient humidity levels, and air quality sensors 347 to detect the presence of pollutants or allergens.

In various embodiments, body sensors 349 may be employed to monitor user-centric parameters, such as heart rate, skin temperature, or galvanic skin response, providing valuable insights into the user's physiological response to the heat therapy. These sensors 349 can communicate with the controller 351 through wired or wireless interfaces (e.g., Bluetooth, Wi-Fi), ensuring seamless data transmission and allowing for real-time adjustments.

According to various embodiments, the controller 351 are sophisticated electronic modules designed to process data from the integrated sensors 341 and adjust the combination device's operational parameters accordingly. In particular implementations, the controller 351 may utilize microcontrollers, application-specific integrated circuits (ASICs), or dedicated heat therapy control ICs to execute complex algorithms and control logic.

In various embodiments, the controller 351 can regulate a range of functions, including, but not limited to, heating element power output and airflow circulation rates. For instance, if the temperature sensors 341 detect a deviation from the setpoint temperature, the controller 351 may dynamically adjust the heating element's power output to maintain a consistent and comfortable temperature.

According to various embodiments, the controller 351 may also feature advanced user interface (UI) components, such as touch-sensitive displays, voice assistants, or mobile app connectivity, allowing users to effortlessly customize their heat therapy experience. Users can select from predefined programs, adjust temperature and humidity levels, or even schedule sessions in advance, all through an intuitive and user-friendly interface.

In particular embodiments, the controller 351 may incorporate machine learning (ML) or artificial intelligence (AI) capabilities, enabling the combination device to learn the user's preferences over time and adapt its operational parameters for an increasingly personalized experience. For example, if a user consistently adjusts the temperature to a specific level during their sessions, the controller 351 can anticipate this preference and automatically adjust the temperature accordingly in future sessions.

According to various embodiments, an innovative inner skeleton design allow the device to transform from a cylindrical shape when facing up to a flat configuration when facing down. In particular embodiments, the inner skeleton may be composed of an engineered framework that includes slotted metal or plastic strips with precision-cut slots, allowing for smooth articulation and flexibility. In particular examples, these strips are constructed from lightweight yet robust materials, such as aluminum or high-strength polymers, to minimize weight while maintaining structural integrity. In some implementations, the inner skeleton incorporates interconnected hinges that connect the slotted strips, facilitating effortless transformation between cylindrical and flat configurations.

According to various embodiments, when in sauna dome mode, the inner skeleton can assume a cylindrical form due to the natural curvature of the slotted strips and interconnected hinges. In particular embodiments, reinforcing members, such as ribs or brackets, are strategically positioned to provide enhanced stability and support to the inner skeleton in its cylindrical configuration. This ensures that the device remains structurally sound, even when subjected to various loads or stresses.

In particular embodiments, when the device is flipped over, the inner skeleton effortlessly transitions to a flat shape due to the mechanical advantage provided by the slotted strips and hinges. In particular embodiments, this transformation is achieved without requiring external inputs or complex actuation mechanisms, thereby simplifying the design and enhancing user convenience.

According to various embodiments, mechanisms such as the glass fiber sheet may be incorporated into the construction of the sauna dome 301. In particular embodiments, in addition to or in place of using an inner skeleton, glass fiber sheets are pre-formed with a latent curvature by leveraging anisotropic material properties to achieve a semicylindrical shape that becomes apparent when the device is transformed into sauna dome mode. This innovative design element can enhance the structural integrity and thermal efficiency of the device.

According to various embodiments, glass fiber sheets may have a number of glass fiber subsheets, utilized in various applications including the sauna dome's structural component, can exhibit anisotropic material properties. In particular embodiments, the sheet's stiffness and flexibility vary significantly depending on the direction in which forces are applied or stresses are induced. According to various embodiments, fibers are often aligned in specific directions to enhance mechanical properties, such as tensile strength or stiffness, in those particular directions. The distribution pattern of glass fibers throughout the sheet can also contribute to anisotropy. Variations in fiber density or clustering can lead to differences in local material properties. In particular embodiments, the glass fiber sheets are oriented such that its stiffer direction (longitudinal) aligns with the primary load paths or stress concentrations in the sauna dome when in sauna dome mode. The glass fiber sheet is configured to lay substantially flat when one side is facing down and rests in a semicylindrical shape when that same side is facing up.

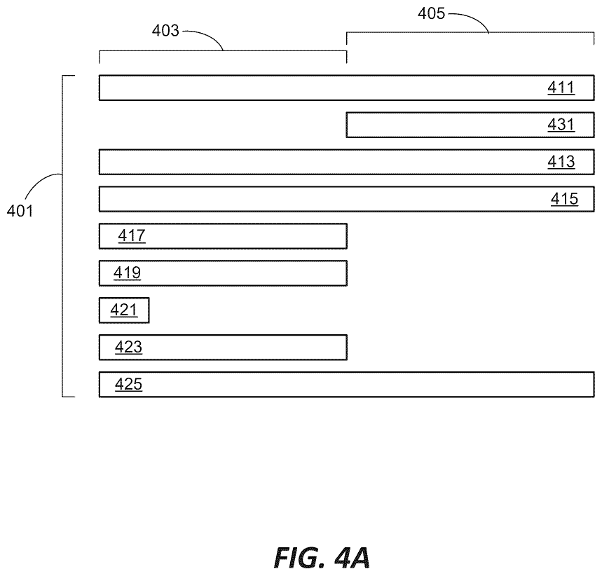

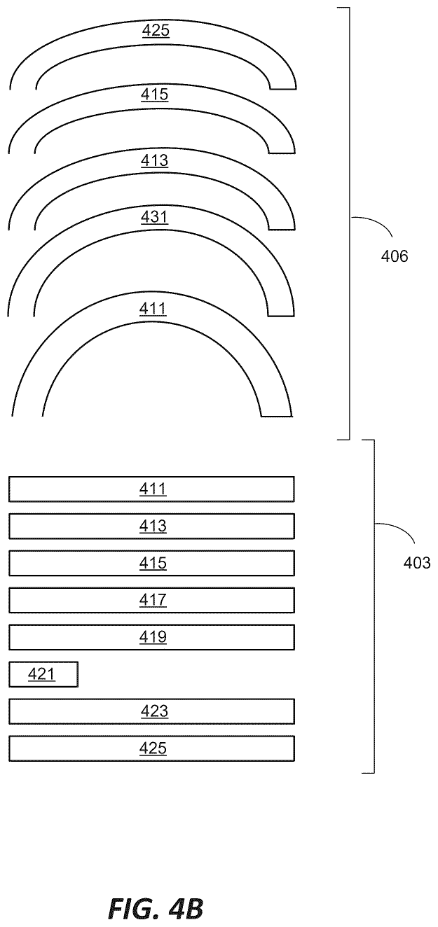

FIGS. 4a and 4b illustrate layers of the combination sauna dome sauna blanket 401 in sauna blanket and in sauna dome modes. According to various embodiments, the portion of the sauna blanket that remains underneath a user when in sauna dome mode is referred to herein as the base portion 403 of the device. The portion of the sauna blanket that is folded over to form the top, curved portion of the device when in sauna dome mode is referred to herein as the dome portion 405. In particular embodiments, the combination device base portion 403 has different layers and materials than the dome portion 405.

The layers that are closest to the user when in sauna dome more are referred to as the innermost layers and the layers that are closest to the exterior when in sauna dome more are referred to as the outermost layers. According to various embodiments, the layers of the base portion 403 from innermost to outermost include oxford fabric with PU 411, heating layer 413 (acupuncture cotton and heat wire and acupuncture cotton), aluminum foil braided fiber 415, acupuncture cotton/T=2 mm 417, energy fabric 419, magnets 421, acupuncture cotton/T=3 mm 423, and PU leather 425.

According to various embodiments, the layers of the dome portion 406 from innermost to outermost include oxford fabric with PU 411, glass fiber sheet (10 pcs) 431, heating layer 413 (acupuncture cotton and heat wire and acupuncture cotton), aluminum foil braided fiber 415, and PU leather 425.

According to various embodiments, the oxford fabric with PU 411 provides a gentle interface with the user's skin while offering thermal insulation and mold resistance. In particular embodiments, the heating layer 413 is a common component in both the base portion 403 and the dome portion 405, positioned to effectively distribute heat throughout the device. This layer includes a sandwiched structure having acupuncture cotton, a heat wire, and additional acupuncture cotton, carefully designed to radiate soothing warmth to the user. The heat wire efficiently warms the surrounding acupuncture cotton layers, ensuring consistent and comfortable heat delivery.

According to various embodiments, both the base portion 403 and the dome portion 406 incorporate an aluminum foil braided fiber layer, designated as 415. This innovative material selection allows for the efficient reflection of radiant heat back towards the user, thereby increasing thermal efficiency and minimizing heat loss. The braided fiber structure enhances durability and flexibility.

In particular embodiments, the base portion 403 includes an additional acupuncture cotton layer, specified as T=2 mm (thickness), denoted as 417. This layer provides supplementary thermal insulation, maintaining a comfortable thickness to ensure optimal warmth without discomfort. According to various embodiments, the base portion 403 features an energy fabric layer, designated as 419, which aims to enhance the user's energy and well-being through proprietary fabric technology. In particular embodiments, the base portion 403 includes embedded magnets, denoted as 421, which facilitate effortless transformation between sauna blanket and dome modes. In particular embodiments, these magnets ensure an unobstructive configuration in either setup, enhancing user convenience.

According to various embodiments, the base portion 403 further includes an additional acupuncture cotton layer, specified as T=3 mm (thickness), designated as 423. This layer provides extra thermal insulation and comfort, contributing to the device's overall performance.

The heating layer 413, situated above the oxford fabric, is a carefully crafted composition of acupuncture cotton, heat wire, and acupuncture cotton, designed to distribute heat evenly throughout the base portion. The heat wire efficiently warms the surrounding acupuncture cotton layers, which in turn radiate soothing warmth to the user. Alternative embodiments might explore the use of different heating elements, such as carbon fiber or thermoelectric materials, or adjust the number of acupuncture cotton layers to modulate heat intensity.

The aluminum foil braided fiber layer 415 plays a crucial role in reflecting radiant heat back towards the user, thereby increasing thermal efficiency and minimizing heat loss. This innovative material selection enables the base portion to retain warmth effectively. In alternative embodiments, this layer could be substituted with other reflective materials, such as Mylar or aluminized polyester, or enhanced by adjusting the braid density for enhanced performance.

Additional thermal insulation is provided by the acupuncture cotton layer 417, which maintains a comfortable thickness of T=2 mm. This deliberate design choice ensures the user experiences optimal warmth without discomfort. Alternative embodiments might involve adjusting the thickness (e.g., T=1.5 mm or T=3 mm) or replacing this layer with alternative insulating materials, such as Thinsulate or Holofill, to achieve varied thermal profiles.

The energy fabric layer 419 is a unique component that aims to enhance the user's energy and well-being through proprietary fabric technology. This might incorporate minerals, essential oils, or bio-ceramic elements, which are believed to promote relaxation and rejuvenation. While the exact mechanisms behind this layer are not fully disclosed, its presence underscores the device's commitment to holistic wellness.

The magnets 421, embedded within the base portion, facilitate effortless transformation between sauna blanket and dome modes, ensuring a secure and stable configuration in either setup. According to various embodiments, the base portion 403 further includes an additional acupuncture cotton layer, specified as T=3 mm (thickness), designated as 423. This layer provides extra thermal insulation and comfort, contributing to the device's overall performance.

In particular embodiments, both the base portion 403 and the dome portion 406 feature polyurethane (PU) leather as their outermost layer, denoted as 425. This durable, water-resistant material protects the underlying layers while maintaining a luxurious feel against the user's skin.

According to various embodiments, the dome portion 406 also includes a glass fiber sheet, comprising ten individual pieces, designated as 431. This structural component provides the necessary rigidity and shape retention for the dome's semicylindrical configuration, ensuring a stable and enclosed space above the user.

Another configuration involves using a hexagonal frame and panels instead of a semicylindrical structure. The sauna dome from the foot and head ends may resemble an edged and cornered structured instead of a rounded or elliptical structure. These panels can be made of a lightweight, insulating material, reducing material usage while maintaining thermal efficiency. The modular design allows for easy disassembly and reconfiguration, providing increased versatility and reduced storage requirements. Furthermore, this approach enables potential customization options, catering to diverse user preferences.

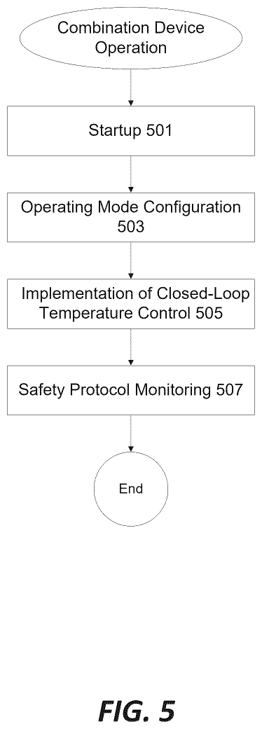

FIG. 5 illustrates a technique for operating the combination sauna dome sauna blanket. According to various embodiments, the control process for the combination sauna dome sauna blanket is initialized at 501. In particular embodiments, the device's default settings are retrieved from memory. Subsequently, the user may be prompted to input their preferred operating parameters via a user interface, such as a temperature knob, digital input, touch screen, voice assistant, or mobile app. These parameters may include, but are not limited to, desired temperature range for a selected sauna mode (dome, blanket, or auto-switching), timer settings for automatic shutdown, and optional energy fabric or auxiliary feature activation.

Upon receiving the user's input, the controller selects and configures the operating mode at 503. According to various embodiments, the controller can generate control signals to adjust temperature, humidity, temperature gradients, etc. In response to the selected mode, the system adjusts various parameters to enhance the user experience. For instance, in sauna dome mode, the system configures the heating elements, airflow circulation, and lighting to create an immersive enclosed sauna environment. Conversely, in sauna blanket mode, the system tailors the heating elements, temperature gradients, and energy fabric (if equipped) for an intimate, contact warmth experience.

According to various embodiments, the controller also implements closed-loop temperature control at 505. In particular embodiments, temperature sensors continuously monitor the device's internal temperature. The heating elements are then dynamically adjusted to maintain the user-set temperature range, ensuring a safe and comfortable experience. Furthermore, overtemperature protection and thermal runaway prevention mechanisms are engaged if necessary to prevent any potential hazards.

In particular embodiments, the controller implements safety features at 507. These features include timer-based automatic shutdown, temperature deviation alerts, and system fault detection. For example, the device powers down after a user-defined period of inactivity or elapsed time, and audible, visual, or haptic warnings are issued if the temperature exceeds predetermined thresholds.

In embodiments where the device is equipped with energy fabric or auxiliary features, the controller activates or deactivates these features based on user input or predefined conditions. The system may also monitor and adjust energy fabric parameters to enhance the user's experience, such as adjusting intensity, frequency, or spectral output.

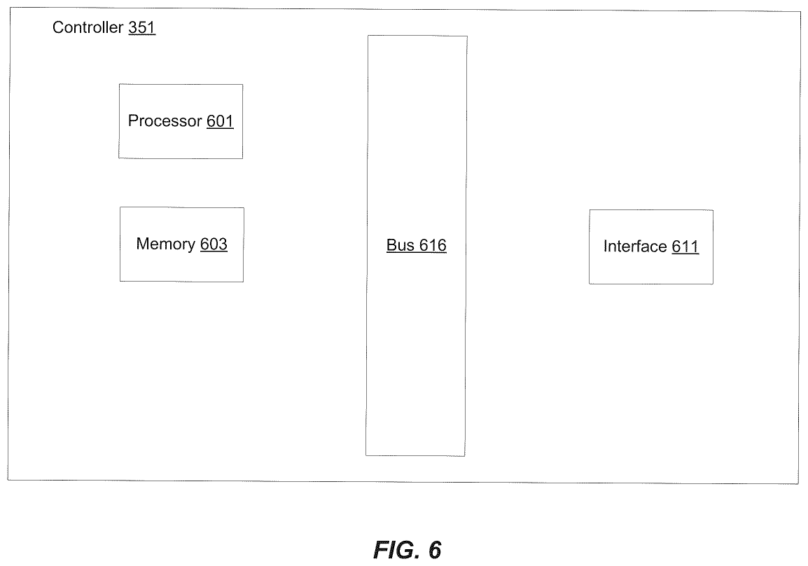

FIG. 6 illustrates one example of a controller, configured in accordance with some embodiments. According to particular embodiments, a controller 351 suitable for implementing particular embodiments of the present invention includes a processor 601, a memory 603, an interface 611, and a bus 616 (e.g., a PCI bus or other interconnection fabric), is configured to control the operation of heaters, as discussed above. Although a controller may include multiple different components, in some examples, a controller may simply be a knob with a variable resistor to control power delivered to heating elements. When acting under the control of appropriate software or firmware, the processor 601 is responsible for receiving inputs and controlling the operation of heaters and heating elements. In some embodiments, system 600 may be integrated in controller 351 discussed above, or may be implemented separately as a stand-alone component that provides functionalities for controller 351. Various specially configured devices can also be used in place of a processor 601 or in addition to processor 601. The interface 611 is typically configured to send and receive data. In some embodiments, such an interface may be configured receive sensor data and/or send control signals to heating elements. In other embodiments, the interface 611 can send packets or data segments over a network.

Particular examples of interfaces supported include Ethernet interfaces, cable interfaces, DSL interfaces, token ring interfaces, and the like. In addition, various very high-speed interfaces may be provided such as fast Ethernet interfaces, Gigabit Ethernet interfaces, ATM interfaces, HSSI interfaces, POS interfaces, FDDI interfaces and the like. Generally, these interfaces may include ports appropriate for communication with the appropriate system components. In some cases, they may also include an independent processor and, in some instances, volatile RAM. The independent processors may control communications-intensive tasks.

According to various embodiments, the system 600 is a controller configured to control the operation of heating elements in accordance with the modes and operations discussed above. The controller may include one or more hardware elements as shown in FIG. 6. In some implementations, one or more of the controller components may be virtualized. For example, a physical server may be configured in a localized or cloud environment. Although a particular controller is described, it should be recognized that a variety of alternative configurations are possible. For example, the modules may be implemented on another device connected to a server.

In the foregoing specification, the invention has been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

What is claimed is:

1. A device comprising:
a flexible, thermally insulating structure configurable between at least two distinct modes of operations, wherein a first mode is a sauna blanket for direct contact with a user and a second mode is a sauna dome for creating a heated air enclosure around the user, wherein the heated air enclosure is substantially semicylindrical;
a controller configured to generate a control signal provided to the flexible, thermally insulating structure, the control signal operable to control operation of the flexible, thermally insulating structure.

2. The device of claim 1, wherein the flexible, thermally insulating structure is a sheet comprising a plurality of layers including a heating element layer and a reflective insulation layer.

3. The device of claim 1, wherein the sauna dome has a width of 20-30 inches and a length of 50-80 inches and a height of 25-35 inches.

4. The device of claim 1, wherein the flexible, thermally insulating structure comprises magnets facilitating easy transformation between the first mode and the second mode.

5. The device of claim 1, wherein the flexible, thermally insulating structure includes a base portion and a dome portion when in sauna blanket mode and when in sauna dome mode.

6. The device of claim 5, wherein the base portion comprises from innermost to outermost:
an oxford fabric with PU leather layer;
a heating element layer;
an aluminum foil braided fiber layer; and
an acupuncture cotton layer.

7. The device of claim 5, wherein the base portion comprises from innermost to outermost:
an oxford fabric with PU leather layer;
a heating element layer
an aluminum foil braided fiber layer;
an first acupuncture cotton layer,
an energy fabric layer;
magnets along one edge,
a second acupuncture cotton layer; and
a PU leather layer.

8. The device of claim 5, wherein the dome portion comprises from innermost layer to outermost layer:
an oxford fabric with PU leather layer;
a glass fiber sheet layer;
a heating element layer; and
a PU leather layer.

9. The device of claim 5, wherein the dome portion comprises from innermost layer to outermost layer:
an oxford fabric with PU leather layer;
a glass fiber sheet;
a heating element layer;
an aluminum foil braided fiber layer; and
a PU leather layer.

10. The device of claim 5, wherein the dome portion comprises a glass fiber sheet with a first side and a second side, the glass fiber sheet configured to lay substantially flat when the first side is facing down and to rest in a semicylindrical shape when the first side is facing up.

11. The device of claim 10, wherein the glass fiber sheet has anisotropic material properties with stiffness and flexibility varying depending on orientation.

12. The device of claim 10, wherein the glass fiber sheet comprises a plurality of glass fiber subsheets.

13. The device of claim 10, wherein the glass fiber sheet is a pre-formed with a latent curvature that becomes apparent when the glass fiber sheet has the first side facing up.

14. The device of claim 10, wherein the glass fiber sheet is not included in the base portion.

15. A combination sauna dome sauna blanket, comprising:
a flexible, thermally insulating cover configurable between at least two distinct modes of operations, wherein a first mode is a sauna blanket for direct contact with a user and a second mode is a sauna dome for creating a heated air enclosure around the user, wherein the heated air enclosure is substantially semicylindrical;
a controller configured to generate a control signal provided to the flexible, thermally insulating cover, the control signal operable to control operation of the flexible, thermally insulating cover.

16. The combination sauna dome sauna blanket of claim 15, wherein the flexible, thermally insulating cover is a sheet comprising a plurality of layers including a heating element layer and a reflective insulation layer.

17. The combination sauna dome sauna blanket of claim 15, wherein the sauna dome has a width of 20-30 inches and a length of 50-80 inches and a height of 25-35 inches.

18. The combination sauna dome sauna blanket of claim 15, wherein the flexible, thermally insulating cover comprises magnets facilitating easy transformation between the first mode and the second mode.

19. The combination sauna dome sauna blanket of claim 15, wherein the flexible, thermally insulating cover includes a base portion and a dome portion when in sauna blanket mode and when in sauna dome mode.

20. The combination sauna dome sauna blanket of claim 19, wherein the base portion comprises from innermost to outermost:
an oxford fabric with PU leather layer;
a heating element layer;
an aluminum foil braided fiber layer; and
an acupuncture cotton layer.
